(19) United States
(12) Patent Application Publication
(10) Pub. No.: US 2021/0299353 A1
(43) Pub. Date: Sep. 30, 2021

(54) HEALTHCARE MONITORING SYSTEM

(71) Applicant: Medisyne Limited, London (GB)

(72) Inventors: Gurdeep MANNU, London (GB); Joao BETTENCOURT, London (GB)

(73) Assignee: Medisyne Limited, London (GB)

(21) Appl. No.: 17/264,196

(22) PCT Filed: Aug. 2, 2019
(86) PCT No.: PCT/GB2019/052185

§ 371 Date: Jan. 28, 2021

(30) Foreign Application Priority Data
Aug. 3, 2018 (GB) 1812653.2

(51) Int. Cl. A61M 5/172; G06N 20/00; G16H 40/67; G16H 50/20; G16H 40/40; G01N 33/493

(52) U.S. Cl. CPC A61M 5/1723; G06N 20/00; G16H 40/67; G16H 10/60; G16H 40/40; G01N 33/493; A61M 2205/18; G16H 50/20

(57) ABSTRACT

A system is disclosed which may be used to infer changes in a patient's condition. The system is arranged to receive at least one input parameter from at least one medical device. The system is configured to infer a change in a patient's condition based on the at least one input parameter and configured to infer the change in the patient's condition using a trained model.

### FIELD

The invention relates to a healthcare monitoring system. Particularly, but not exclusively, the invention relates to a healthcare monitoring system which is configured to determine a change in the condition of a patient based on an output biofluid measurement value. Further particularly, but not exclusively, the invention relates to a healthcare monitoring system which uses the techniques of artificial intelligence to infer changes in a patient's condition.

### BACKGROUND

Biofluids are liquids which are within the bodies of living things, such as people and animals. One example of a biofluid, urine, is often taken from patients during examination in a medical environment, such as a hospital. Typically, a urinary catheter is used to drain the patient's urine into a catheter bag.

Urine can be used to indicate the prevalence of a number of different conditions and changes in urine production can be used to determine the presence of, among other things, dehydration, infection, trauma, urinary tract obstruction, functioning of transplanted organs such as urinary amylase and pH in pancreas transplantation and the presence of some forms of medication in the body of a patient. Examples of such medications are non-steroidal anti-inflammatory drugs, high blood pressure medication and gentamicin.

Abnormally high urine production, known as polyuria, can be indicative of diabetes, hyperthyroidism, Conn's disease, hyperglycaemia, postural orthostatic tachycardia syndrome and haemochromatosis.

Abnormally low urine production, known as oliguria, can be indicative of dehydration, sepsis, kidney failure, hypovolemic shock, pre-eclampsia and diabetic ketoacidosis.

It is therefore important to be able to measure the output of biofluids such as urine, as they can indicate deterioration of a patient's condition before other signs or symptoms are apparent.

Aspects and embodiments were conceived with the foregoing in mind.

### SUMMARY

Aspects relate to systems for the measurement of biofluids. A biofluid is a biological fluid which may be excreted from a human or animal, secreted by a human or animal, obtained from a human or animal with a needle or other device or developed in a human or animal by a pathological process associated with the human or animal.

Examples of biofluids may include urine, sweat, breast milk, bile, blood, cerebrospinal fluid, ascitic fluid, blister fluid, pus or other exudate, cyst fluid, surgical drain output, nasogastric tube output, colostomy output, ileostomy output and other stoma output.

Viewed from a first aspect there is provided a healthcare monitoring system, the system arranged to receive at least one input parameter from at least one medical device, wherein the system is configured to infer a change in a patient's condition based on the at least one input parameter, wherein the system is further configured to infer the change in the patient's condition using a trained model.
The system may be configured to generate an alert based on the inference of a change in the patient's condition.

A system in accordance with an aspect receives parameters from at least one medical device and infers a change in patient condition based on a trained model by feeding the parameters into the trained model—which then uses rules on parameters to determine whether the parameters are indicating the need for an alert to indicate a change in condition.

That is to say, the system may infer a change based on a change in a parameter and that parameter is then used as input to a trained model which infers whether a change has taken place

Viewed from a further aspect, there is provided a biofluid measurement system, the system arranged to receive a flow of biofluid from a biofluid source on a patient, the biofluid measurement system comprising a biofluid reservoir coupled to the biofluid source for receiving the flow of biofluid from the patient, and at least one biofluid sensing arrangement configured to determine the amount of the biofluid in the biofluid reservoir to provide at least one output biofluid measurement value indicative of the amount of the biofluid in the biofluid reservoir, wherein the system is configured to infer a change in the patient's condition based, at least in part, on the at least one output biofluid measurement value; and the system is further configured to generate an alert based on the inference of a change in the patient's condition.

A system in accordance with an aspect determines the need for an alert based on biofluid output from a patient.

The system may be further configured to communicate the alert to a device, which may be a remote device, to enable a user to be notified of the inference of a change in the patient's condition.

The amount of biofluid in the biofluid reservoir may comprise the volume of the biofluid in the biofluid reservoir.

This means that the alert may be transmitted to a practitioner who is then alerted to the change in the patient's condition.

The biofluid reservoir may comprise a tank arranged to receive the flow of biofluid, wherein the tank may be arranged to form an enclosure for the storage of the biofluid received from the patient.

The tank may be detachably mounted to the biofluid reservoir and may be aseptic.

The tank may comprise an array of ultraviolet light emitting diodes mounted on a wall of the tank, wherein the array of ultraviolet light emitting diodes is arranged to disinfect the biofluid in the tank. The effect of this is that the biofluid is disinfected whilst it is being measured in the tank.

The advantage of using ultraviolet light emitting diodes is that they eliminate the production of corrosive metal and disinfection by-products.

The biofluid sensing arrangement may be configured to determine the amount of biofluid in the biofluid reservoir by weighing the biofluid reservoir.

The biofluid sensing arrangement may be configured to determine the amount of biofluid in the biofluid reservoir using a stretch mechanism mounted between the biofluid source and the reservoir.

The biofluid sensing arrangement may be configured to determine the amount of biofluid in the biofluid reservoir using a weighted mechanism mounted between the biofluid source and the reservoir.

The biofluid sensing arrangement may comprise at least one ultrasonic sensor mounted to the biofluid reservoir, wherein the at least one ultrasonic sensor is configured to determine the amount of biofluid in the biofluid reservoir by reflecting ultrasonic waves off the surface of the biofluid in the biofluid reservoir.

Using an ultrasonic sensor to determine the amount of biofluid in the biofluid reservoir is advantageous as ultrasonic sensors can be made generally smaller than other sensors

The biofluid reservoir may be arranged to enable the biofluid to be drained out of the biofluid reservoir. The effect of this is that it is easy to clean the biofluid reservoir.

The biofluid reservoir may comprise a valve actuatable between an open and closed position, wherein in the open position the valve enables the biofluid to be drained out of the reservoir and in the closed position the valve prevents the biofluid from being drained out of the reservoir.

The biofluid reservoir comprises an adaptor for the coupling of a detachable container for receiving the biofluid during drainage from the biofluid reservoir.

The detachable container may further comprise a mounting mechanism to enable the detachable container to be securely mounted to the biofluid reservoir whilst the detachable container is coupled to the adaptor whilst the biofluid is being drained into the detachable container.

The mounting mechanism may be a clip arranged to securely couple the detachable container to a corresponding mechanism on the reservoir which enables the detachable container to be retained in position whilst the biofluid is being drained into the detachable container.

The detachable container may comprise a disposable bag.

The system may be configured to infer a change in the patient's condition based on a trained model.

The system may be configured to infer a change in the patient's condition using a knowledge base and rules determined using the knowledge base.

The system may be further configured to determine a trained model from the application of machine learning techniques to historical data, wherein the trained model may be used to infer a change in the patient's condition.

The system may be further configured to determine a trained model from the application of statistical inference techniques to historical data, wherein the trained model is used to infer the change in the patient's condition.

The historical data may comprise fields relating to at least one of physiological, biochemical or biometric output, such as, but not limited to, the chemical composition of biofluid output, frequency of biofluid output, blood pressure, body temperature, oxygen saturation and respiratory rate.

The fields may pertain to a demographic group which may be defined by at least one of a biometric, diagnostic test result, condition, an illness, gender or age.

Rules may be modified by any of the fields. One example may be where the fields relate to blood test results. That is to say, if the blood test results indicate an abnormal blood urea nitrogen level then this is an indicator of kidney function and this will likely influence what would be considered to be a normal level of urine output.

Rules may be modified by data related to imaging. Indeed, if the patient has recently had a CT scan with contrast, this can damage the kidneys which would change the normal levels or urine output.

The system may be configured to determine at least one rule from the trained model.

The rule may define a danger zone on at least one parameter, wherein the danger zone defines a threshold on the at least one parameter to indicate when a measurement for that parameter indicates deterioration in the patient.

The system may be configured to infer a change in the patient's condition responsive to a determination that the at least one parameter is indicating deterioration in the condition of the patient.

The system may, responsive to inferring a change in the patient's condition, be configured to initiate a response action designated by the rule.

The response action may be an instruction to a connected device.

The response action may be a message to a remote device to indicate the need for an increased frequency of monitoring of the patient and/or to alert healthcare staff to review the change in a given patient's parameters

The response action may be a message to a remote device to indicate suggested guidance/action by healthcare staff in response the change in a given patient's parameters.

The response action may be a message to a remote device to indicate the need to maintain a currency frequency of monitoring of the patient.

The response action may be a request to a fluid bolus mechanism to increase or decrease the amount of fluid delivered to the patient.

The response action may be a request to a fluid bolus mechanism to increase or decrease the rate of fluid delivered to the patient.

The fluid bolus mechanism may, responsive to receiving the request, implement the request and delivers the requested amount or rate to the patient.

The fluid bolus mechanism may comprise a pressure cuff to enable an operator to mechanically increase the rate of fluid delivery from the fluid bolus mechanism to the patient by pressing the cuff.

The fluid bolus mechanism may comprise a syringe arranged to deliver a specified amount of fluid bolus to the patient.

The system will use parameters in addition to urine output, blood pressure, heart rate, oxygen saturation, respiratory rate and temperature such as continuous central venous pressure monitoring in order to stimulate outputs such as remote alerts and/or fluid bolus.

The fluid bolus mechanism may comprise an electric motor arranged to enable the delivery of uniform amounts of fluid bolus to the patient.

The remote device may be selected by the trained model based on the responsiveness of the operator designated to the remote device.

The system may be configured to infer a change in the patient's condition based on a trained model by:
comparing the received output biofluid measurement value to a rule determined by the trained model, wherein the rule defines a danger zone for the output of the biofluid;
infer a change in the patient's condition by determining a transition of the output of the biofluid into the danger zone.

The trained model may be determined using historical data corresponding to the disease or condition suffered by the patient. The trained model may be determined using simulation data corresponding to the disease or condition suffered by the patient.

The trained model may be based on the demographic status of the patient or on the medical history of the patient.

The trained model may be determined using a machine learning approach wherein the trained model comprises a plurality of rules which are determined using a combination of machine learning techniques.

Viewed from a second aspect, there may also be provided a biofluid measurement system, the system arranged to receive a flow of biofluid from a biofluid source in or on a patient, the biofluid measurement system comprising a biofluid reservoir coupled to the biofluid source for receiving the flow of biofluid from the patient; and at least one biofluid sensing arrangement configured to determine the amount of the biofluid in the biofluid reservoir to provide at least one output biofluid measurement value indicative of the amount of the biofluid in the biofluid reservoir; wherein the system is configured to infer a change in the patient's condition based, at least in part, on the at least one output biofluid measurement value; and the system is further configured to automatically generate a response action based on the inference of a change in the patient's condition.

A system in accordance with the second aspect provides a biofluid measurement system which automatically generates a response action if the output biofluid indicates a change in the patient's condition. The response action may be an alert to a responsible practitioner or an instruction to a medical device.

Viewed from a third aspect, there may also be provided a biofluid measurement system, the system arranged to receive a flow of biofluid from a biofluid source in or on a patient, the biofluid measurement system comprising: a biofluid reservoir coupled to the biofluid source for receiving the flow of biofluid from the patient; and at least one biofluid sensing arrangement configured to determine the amount of the biofluid in the biofluid reservoir to provide at least one output biofluid measurement value indicative of the amount of the biofluid in the biofluid reservoir; wherein the system is configured to infer a change in the patient's condition based, at least in part, on the at least one output biofluid measurement value; and the system is further configured to generate an instruction for a medical device responsive to inferring a change in the patient's condition.

A system in accordance with the third aspect provides a biofluid measurement system which automatically generates an instruction for a medical device if the output biofluid indicates a change in the patient's condition. Example medical devices may be a fluid bolus mechanism which may be instructed, by the instruction, to alter the delivery of at least one fluid bolus.

### DESCRIPTION

An embodiment in accordance with the aspect will now be described by way of example only and with reference to the following drawings in which:

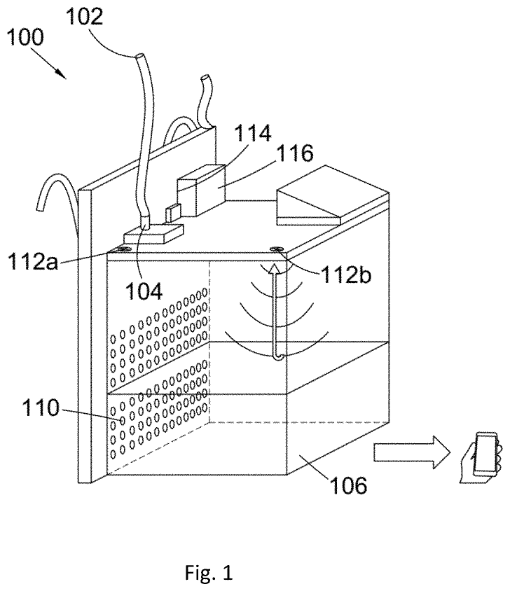

FIG. 1 provides an aspect view of a urine measurement system in accordance with the embodiment;

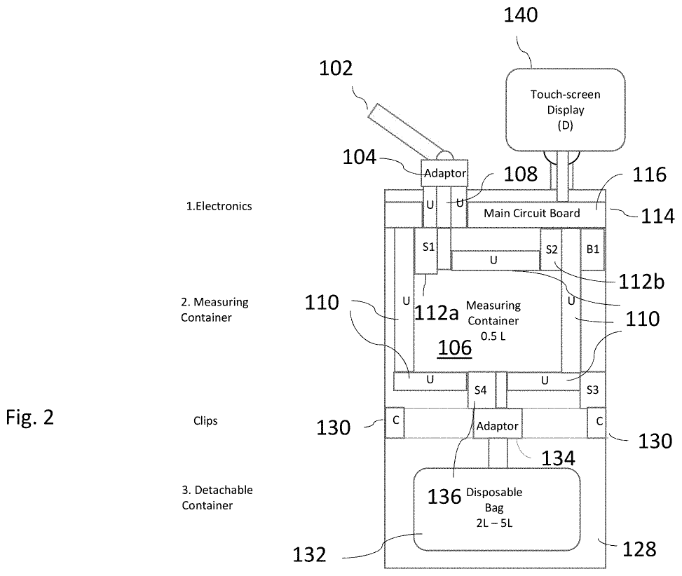

FIG. 2 provides a schematic view of a urine measurement system in accordance with the embodiment;

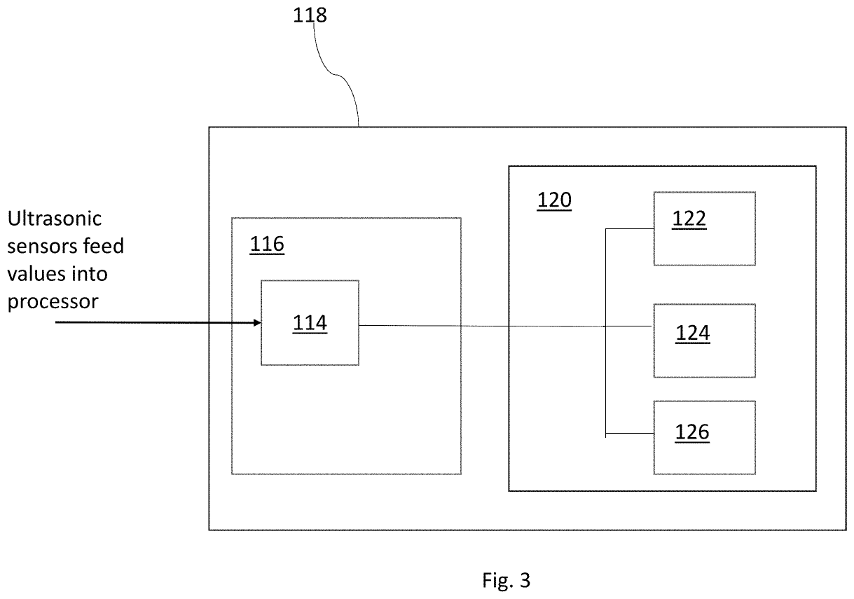

FIG. 3 schematically illustrates a biofluid analytics component in accordance with the embodiment;

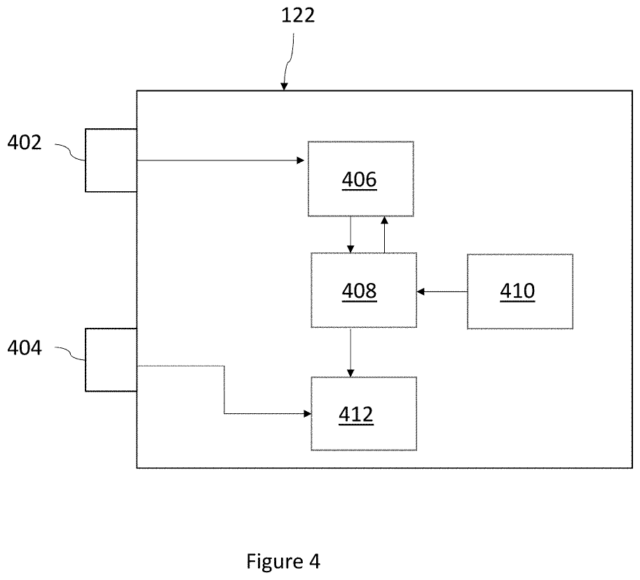

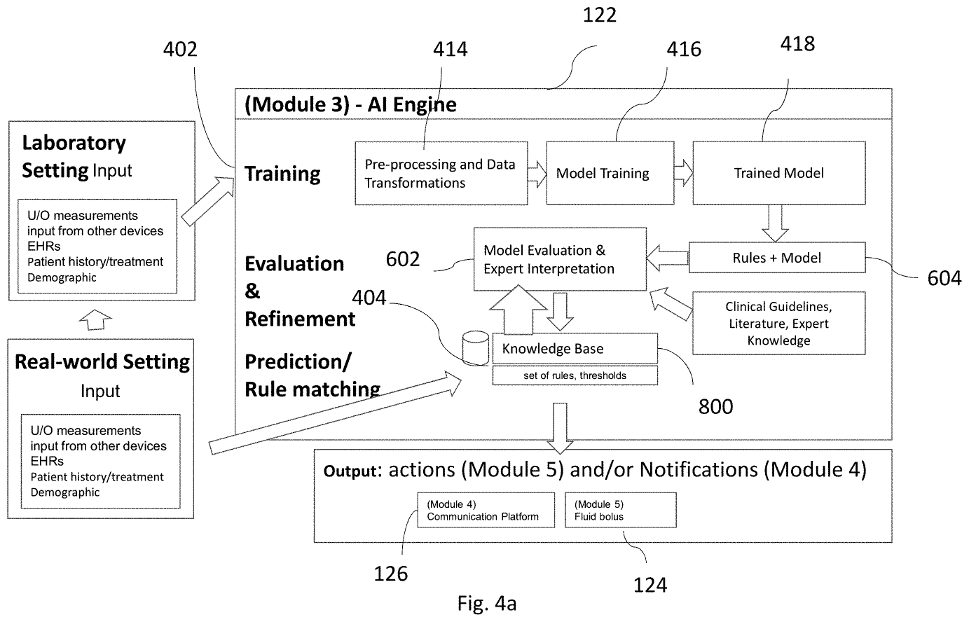

FIG. 4 is a schematic diagram which illustrates the components of an artificial intelligence module in accordance with the embodiment;

FIG. 4a is a schematic diagram which illustrates how the artificial intelligence module interacts with the other modules in the biofluid analytics component;

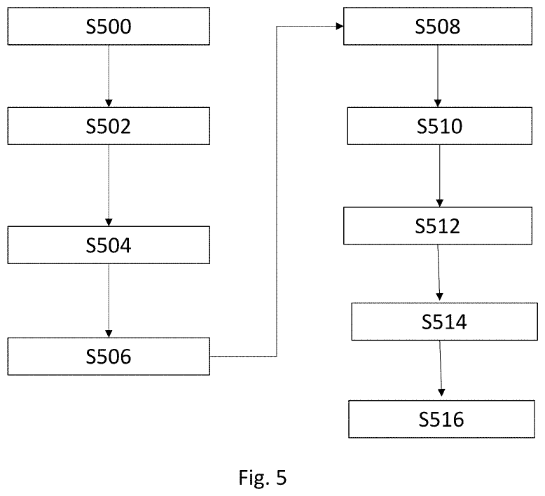

FIG. 5 is a flow diagram illustrating the steps taken by the training component to determine a training model using training data provided from the laboratory;

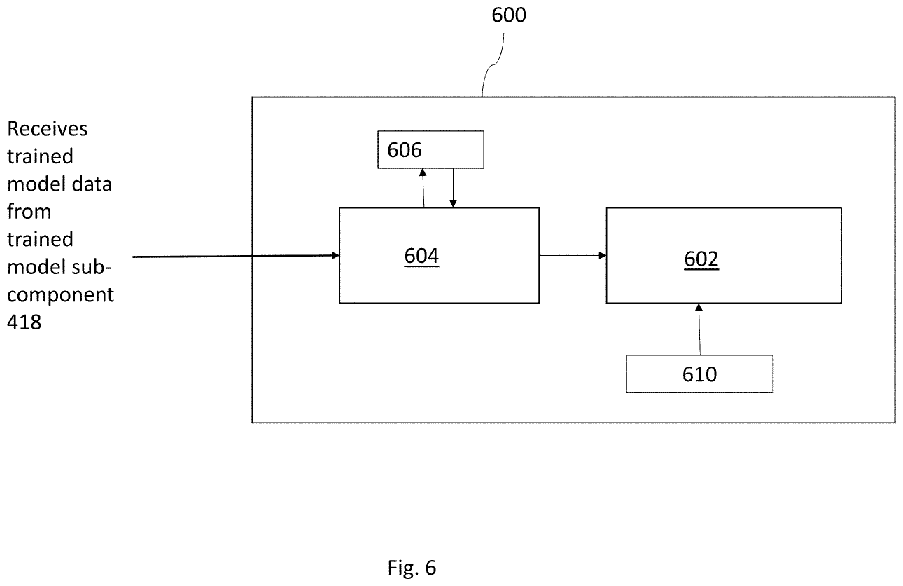

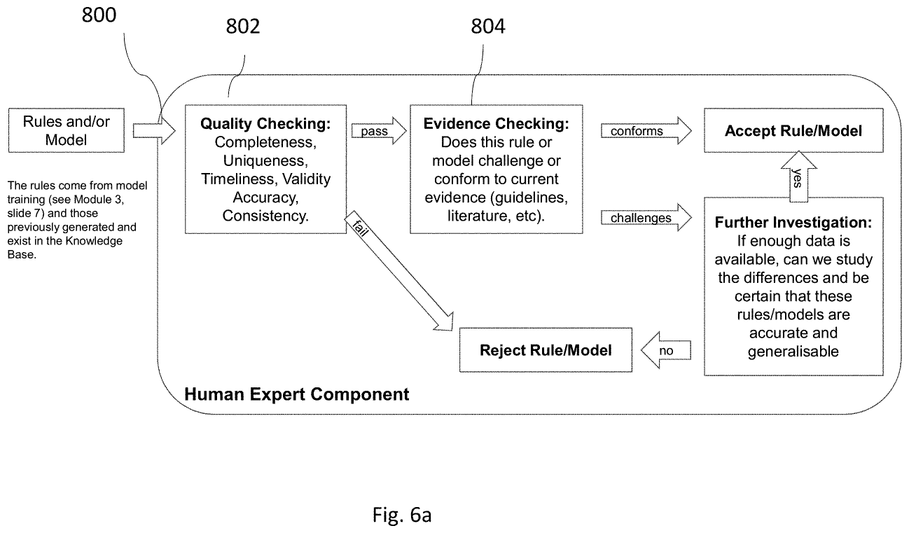

FIG. 6 is a schematic diagram which illustrates the components of an evaluation and refinement component in accordance with the embodiment;

FIG. 6a is a schematic diagram illustrating the steps taken to evaluate the trained model and rules by the model evaluation and interpretation sub-component;

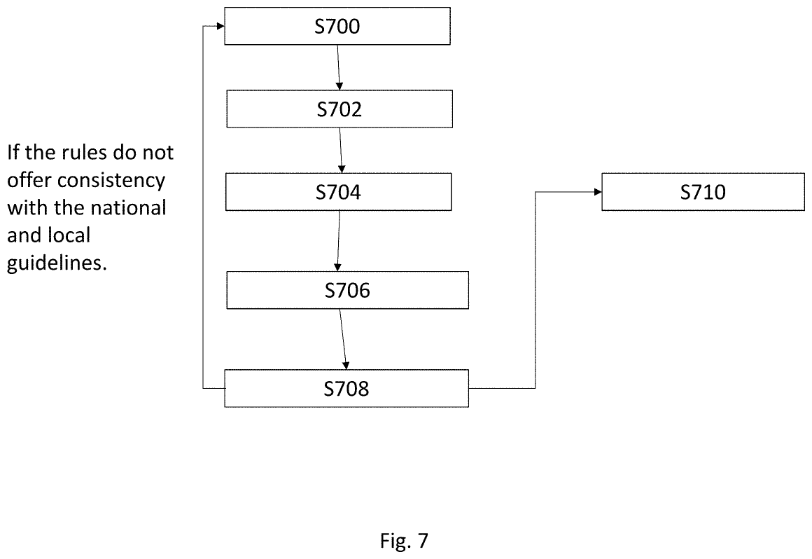

FIG. 7 is a flow diagram illustrating the steps taken by the evaluation and refinement component in accordance with the embodiment;

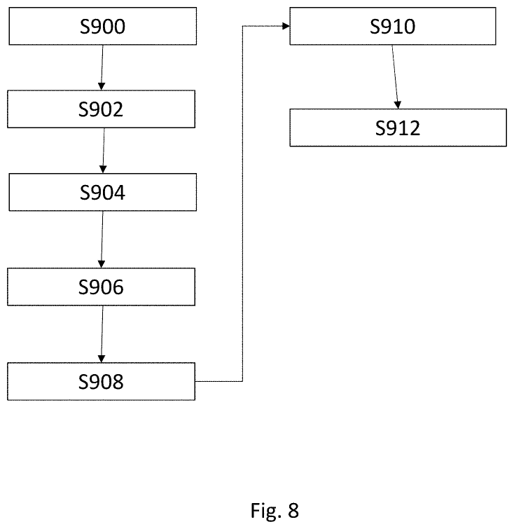

FIG. 8 is a flow diagram illustrating the generation of an alert in accordance with the embodiment; and

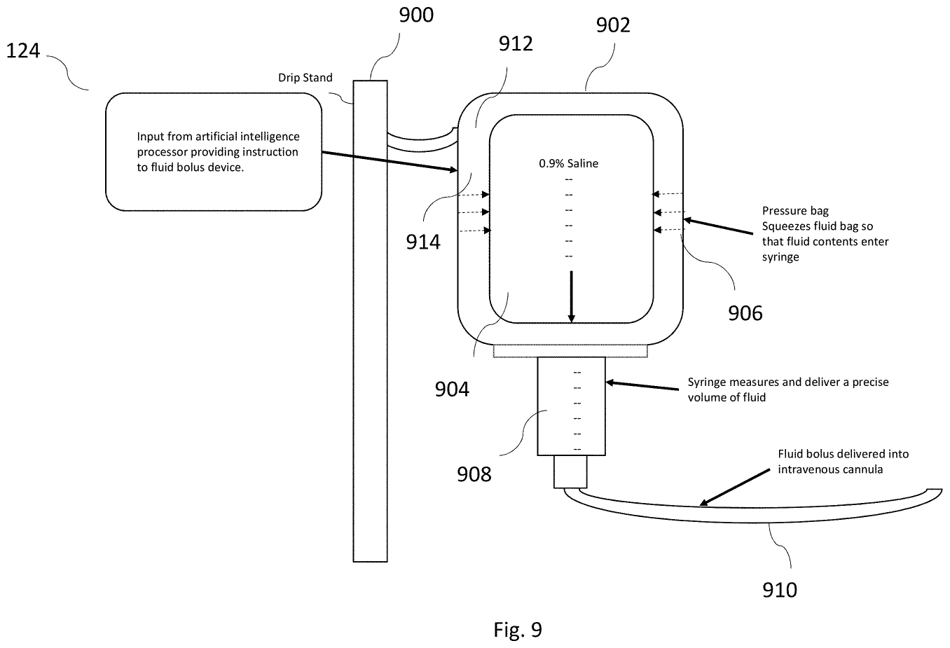

FIG. 9 is a schematic of an automated fluid bolus mechanism in accordance with the embodiment.

We illustrate with reference to FIG. 1 and FIG. 2 a system 100 which is to be used in the measurement of urine. Urine is used only as an example of a biofluid which can be measured by a system within the embodiment. It will be understood that other biofluids can also be measured using the principles hereinafter described.

System 100 receives a flow of urine from a patient through a urinary catheter 102 which is attached to a patient. An example of such a catheter is the Foley catheter. The urinary catheter 102 is coupled to an adaptor 104 which enables the urine to flow from the urinary catheter 102 into a measuring container 106 through a tube 108 which leads from the adaptor 104 into the measuring container 106. The tube 108 may be arranged to feed urine into the bottom of the measuring container 106 to avoid unsightly splashing or wave generation. This ensures the modesty of the patient is maintained whilst they are connected to the system 100.

The flow of urine into the measuring container 106 causes the urine to fill the measuring container 106 where the urine is stored for measurement and analysis by the system 100.

That is to say, the measuring container 106 forms a tank which encloses the urine whilst it is being measured.

Arrays of ultraviolet light-emitting diodes 110 are attached to the walls of the measuring container 106. In one embodiment, only a single array of ultraviolet light emitting diodes may be attached to a wall of the measuring container 106. In other embodiments, arrays of ultraviolet light emitting diodes 110 may be attached to each wall of the measuring container 106.

The use of arrays of ultraviolet light emitting diodes 110 prevent infection of the urine and kills any germs that may be present in the urine. This helps to maintain an aseptic urine reservoir in the measuring container 106.

Ultrasonic sensors 112a and 112b are attached to the roof of the measuring container 106. The ultrasonic sensors 112a and 112b emit ultrasonic waves in the direction of the urine in the measuring container 106.

The measuring container 106 may additionally comprise sensors to enable the system to monitor chemical composition of the urine, temperature of the urine and any other parameter related to the constitution of the urine.

The ultrasonic sensors 112a and 112b are also arranged to detect the reflection of the ultrasonic waves that are emitted in the direction of the urine in the measuring container. That is to say, the ultrasonic waves are reflected by the urine and so the level of the urine in the measuring container affects the time taken for an ultrasonic wave to return to the ultrasonic sensor. This means that the time taken for an ultrasonic wave to return to the ultrasonic sensor is indicative of the volume of urine in the measuring container 106.

The ultrasonic sensors feed back to a processor 114 on a main circuit board 116 a reading indicative of the time it takes for an ultrasonic wave to be reflected by the urine in the measuring container 106.

The processor 114 is configured to determine from the reading the amount of urine in the measuring container 106. That is to say, the processor 114 retains a stored value which is an expected return time of an ultrasonic wave in an empty measuring container 106. The processor 114 can therefore determine, by comparing the reading from the measuring container 106 to the expected return time of an ultrasonic wave in an empty measuring container 106, how much urine is in the measuring container 106.

Alternatively, the amount of urine in the measuring container 106 may be determined by weighing the measuring container 106.

For example, if, say, for a 5 litre measuring container 106, the expected return time is 5 milliseconds for an empty container and the reading that is fed back to the main circuit board 116 is 2.5 milliseconds, the processor 114 is configured to determine that the amount of urine in the measuring container 106 is 2.5 litres. Similarly, if the reading that is fed back to the main circuit board 116 is 1 millisecond, the processor 114 is configured to determine that the amount of urine in the measuring container 106 is 4 litres.

The processor 114 forms part of a biofluid analytical component 118 which also comprises a computer 120 which comprises an artificial intelligence module 122, a fluid bolus module 124 and a communications module 126. This is illustrated in FIG. 3.

The processor 114 feeds the output urine measurement value into the artificial intelligence module 122 which will now be described.

This is an example of a parameter being received by the system 100 from a medical device which delivers a parameter to the system indicative of the patient's urine output.

A touch-screen display 140 is also provided to enable operatives to enter data into the system. This data may be to retrieve the electronic health record of the patient attached to the catheter 102.

The system 100 further comprises a detachable drainage component 128 which is attached to the measuring container 106 via clips 130 placed at each corner of the detachable drainage component 128.

The detachable drainage component 128 contains a disposable bag 132 of equivalent volume to the measuring container 106. The disposable bag 132 is attached to an adaptor 134 on the underside of the measuring container 106.

A valve 136 is positioned at the bottom of the measuring container 106. The valve moves between an open position in which it enables drainage of the urine from the measuring container 106 into the disposable bag 132 and a closed position in which this drainage is prevented. The movement of the valve may be electronically actuated.

The effect of this is that the urine can be drained at frequent intervals during treatment or after treatment has been completed.

The system 100 uses machine learning techniques to build a knowledge base which is used to make predictions based on the urine output of the patient. These techniques will now be described.

The artificial intelligence module 122 and its interaction with laboratory data and real-time data will now be described with reference to FIGS. 4, 4a and 5 as we illustrate how the principles of machine learning and statistical inference are used to build a knowledge base for the system 100. That is to say, the artificial intelligence module is configured to be “artificially intelligent” in that it uses rules that are generated by the application of machine learning and statistical inference to laboratory data to training data. The training data is formed from laboratory data and numerical simulation data. As will be illustrated below, the generation of these rules allows the system 100 to generate recommended actions when a patient's condition starts to deteriorate.

The artificial intelligence module 122 comprises a laboratory input interface 402, a biofluid measurement interface 404, a training component 406, an evaluation and refinement component 408, a clinical guidance component 410 and a prediction component 412. Each of these components comprises further sub-components which will be described below.

The laboratory input interface 402 is configured to receive data input from the laboratory environment. This may also include numerical simulation data. The data received through the laboratory input interface 402 is communicated to the training component 406 using any suitable data transmission means.

The training component 406 is configured to transmit data to the evaluation and refinement component 408 using any suitable data transmission means. The evaluation and refinement component 408 is configured to receive data from the evaluation and refinement component 408 using any suitable data transmission means.

The clinical guidance component 410 is configured to transmit data to the evaluation and refinement component 408 using any suitable data transmission means.

The evaluation and refinement component 408 is configured to transmit data to the prediction component 412 using any suitable data transmission means.

The prediction component 412 is configured to receive input from the biofluid measurement interface 404.

A schematic of how artificial intelligence module 122 interacts with the other parts of the biofluid analytics component 118 is provided into FIG. 4a.

The training component 406 will now be described by reference to FIG. 5 where machine learning principles are used to build an artificially intelligent system 100 by the analysis of training data to determine a training model that can be applied to the treatment of patients who are attached to the system 100.

The training component 406 receives training data in a step S500 through laboratory interface 402 in the form of historical data which has been taken in a medical research laboratory or using a numerical simulation, i.e. a made-up data-set. The values that make up the training data will ultimately be subject of a machine learning approach to draw inferences from the data about the danger zones that a patient can experience and the actions that should be taken if necessary when the patient experiences those danger zones. However, some pre-processing and transformation of the training data is necessary to place the training data into a form which will enable it to be fed into the machine learning approach which we will describe.

The data pre-processing and transformation sub-component 414 applies standard techniques of data cleansing, feature selection and dimensionality reduction to the training data in a step S502.

The techniques of data cleansing that are particularly suitable are data normalisation to rescale the respective variables, outlier detection and removal to avoid unnecessary skewing of the training data, data completeness checks and editing of the data using missing data detection and data imputation techniques.

The techniques of feature selection and dimensionality reduction reduce the number of dimensions in the data to remove redundant information to create a training dataset which is more homogenous and of greater value to the training component 406. Feature selection and dimensionality reduction identify the components of the data which are discriminative. Removal of components of data which will not be discriminative means that computational time will not be taken enough by computations on unnecessary dimensions of the data. Effectively, a manifold is formed between the training data which is received in step S500 and the data which is used to form the model.

Specifically, the training data is made up of fields containing values pertaining to urine volume output, the time over which the urine volume output was excreted, the composition of the urine, heart rate, blood pressure, temperature, oxygen saturation of the blood, respiratory rate and a time component. The training data would also comprise fields relating to demographics, medical conditions and medical treatments, often referred to as an electronic health record.

The training data would also include data which relates to treatments that have been historically applied to conditions which have been caused by increases or decreases in urine output, heart rate, blood pressure, temperature, oxygen saturation of the blood and respiratory rate and how the treatments affect those parameters.

The training data would also include historical records of the monitoring that has taken place responsive to increases or decreases in urine output, heart rate, blood pressure, temperature, oxygen saturation of the blood, respiratory rate.

That is to say, the training data may comprise historical data, say, for urine volume output, the time over which the urine volume output was excreted, the composition of the urine, heart rate, blood pressure, temperature, oxygen saturation of the blood and respiratory rate for all patients who are Latin American males aged 26 to 35 with diabetes, for example.

Alternatively, the training data may comprise urine volume output, the time over which the urine volume output was excreted, the composition of the urine, heart rate, blood pressure, temperature, oxygen saturation of the blood, respiratory rate for all patients who are white Caucasian females aged 55 to 65 with low blood pressure, for example.

The training data may include historical data related to patients with chronic renal failure, sepsis, heart failure, kidney disease, acquired immunodeficiency syndrome, dengue fever, deep vein thrombosis, keratitis, osteoarthritis, whooping cough or any other disease and the respective demographic groups that may or may not have suffered from those diseases. Provided the data on a disease or condition exists then it can be fed into the training component 406 and the following techniques can be applied to the data.

After the application of data cleansing, feature selection and dimensionality reduction to the training data in step S502, the data pre-processing and transformation sub-component 414 outputs a pre-processed and transformed dataset which is transmitted to a model training sub-component 416 in a step S504.

In step S506, the model training sub-component 416 applies techniques of classification, association, pattern mining, clustering, process mining, statistical inference, neural networks and reinforcement learning to the pre-processed and transformed dataset. These techniques may be applied individually or in combination. The model training sub-component 416 also applies techniques such as decision tree pruning, cross-validation and other quality checking techniques used to evaluate the quality of a model. If the model does not meet expected quality requirements then a human may intervene to investigate, correct and to restart the process if necessary.

Classification techniques for example, may include decision trees, classification trees and regression trees or a C4.5 algorithm for rule based classification. Application of these classification techniques enable the data to be classified into say, data relating to low-risk patients, data relating to medium risk patients and data relating to high-risk patients.

The application of classification techniques means that the output trained model (discussed below) identifies data which is indicative of the need for an alert. However, the need for an alert for some demographics may be different from others.

For example, if the data relates to Latin American males aged 26 to 35 with diabetes then the risk profiling would be different from Latin American males aged 26 to 35 without diabetes as diabetes will influence urine output.

Association and pattern mining techniques establish connections in the pre-processed and transformed dataset to find patterns that occur together. One example may be a connection between a patient who has experienced shock and a low volume of urine over a time period which is relatively close to when they experienced the initial shock. Another example may be a connection between patients who are known heroin users and the presence of metabolites associated with heroin usage in their urine. Association and pattern mining techniques may comprise sequential pattern mining, the a priori algorithm and the prefixspan algorithm.

Clustering techniques may group subsets of the pre-processed and transformed data based on their similarities such as, for example, high heart rate, high respiratory rate, high body temperature and low blood pressure and low urine output may be clustered together as a subset of the data. One of the techniques used for clustering may be the K-means clustering algorithm.

Process mining techniques may establish how common sequences of events have occurred and led to poorer or unexpected outcomes. An outcome maybe that the administration of a specific pharmaceutical composition may have an adverse effect on the urine output of a patient with a specific medical condition.

Statistical inference techniques may establish correlations, dispersions and associations in the pre-processed and transformed data set. These techniques may include, for example, regression analysis, generalised linear modelling and significance tests. For example, regression analysis applied to the transformed and pre-processed data-set may determine that there is cut-off threshold for the urine output volume (in millilitres per hour) due to the trends of one or more patients with a similar profile, i.e. similar demographic status, clinical presentation and other relevant parameters.

Neural networks can be applied to the pre-processed and transformed dataset. Neural network techniques enable the model training sub-component 416 to determine the presence of certain tasks in the pre-processed and transformed dataset and the effect of those tasks by association with changes in other aspects of the data.

One example would be the determination of an association between an alert transmitted to a medical practitioner and the feedback provided from that practitioner. A specific example may be that an alert sent around 8:30 in the morning is not a good time to alert a specific practitioner as they are not in the building at that time. This would lead to an association between alerting that practitioner at 8:30 and the need to alert a second practitioner as the first practitioner is unresponsive.

Reinforcement learning techniques may also be applied to the pre-processed and transformed dataset. This could involve the application of techniques such as Markov decision processes, Monte-Carlo methods and temporal difference methods which determine associations between the external environment and how situations link to actions. One example could be that the administration of a particular treatment leads to further complications when that treatment is given to a minor or, for example, an elderly person.

The application of the techniques in step S506 provide an output in the form of a trained model.

The trained model can take a number of forms depending on the output of the techniques applied in step S506.

One example form may be a multi-variate normal distribution wherein the heart rate, respiratory rate, body temperature and urine output are each normally distributed with a respective mean heart rate and standard deviation of the heart rate, mean respiratory rate and standard deviation of the respiratory rate, mean body temperature and standard deviation body temperature and mean urine output and standard deviation urine output.

Another form of trained model may be a linear relationship (determined using linear regression) between shock and low urine output. This connection has been determined because the application of the machine learning approach has determined that historically there has been a connection between shock and low urine output. This form of trained model will be particularly useful when the patient has suffered from shock as it indicates that shock tends to lead to lower urine output—which means that low urine output is more expected than it is from patients who have not suffered from shock.

The two examples of trained models described above may be used in combination. In this instance, it would be useful where the patient was a Latin American male aged 26 to 35 with diabetes who had suffered from shock. The reason for this is that the thresholds on urine output can be adjusted appropriately as the danger zone on urine output is likely to be different.

This means that the multi-variate normal distribution described above can be used to identify danger zones for specific sets of patients by using the machine learning approach to draw connections and determine rules which are found in the data.

In the example of Latin American males aged 26 to 35 with diabetes, the trained model may draw a connection between this demographic and a combination of the random-variables which form the multi-variate, normally distributed training model transmitted to the trained model sub-component 418. Similarly, this connection may be specific only to Latin American males aged 26 to 35 with diabetes and not be applicable to persons outside of this demographic.

The connection may say, for example, that a specific combination of thresholds in the heart rate, respiratory rate, body temperature and urine output are indicative of a serious deterioration in the health of a patient within this demographic. This is because the historical data has suggested that this specific combination of thresholds has, in the past, led to a serious deterioration in the health of previous patients within this demographic group.

The data representing the trained model is transmitted from the model training sub-component 416 to the trained model sub-component 418 in a step S508.

In using the machine learning approach described in respect of steps S500 to S508 to produce a trained model, the trained model transmitted to the trained model sub-component 418 in step S508 has been trained on the laboratory data which was provided to the pre-processing and data transformation sub-component 414 in step S500. That is to say, the training model is based on the historical data provided to the system.

However, the trained model is not useful until it has been tested to ensure the inferences drawn by the trained model are valid. The trained model sub-component 418 retrieves a data file from local storage in a step S510. The data file is structured similarly to the data file provided to the pre-processing and data transformation sub-component 414 in step S500.

The data file contains further data which has not been previously processed by the pre-processing and data transformation sub-component 414 or the model training sub-component 416. The trained model is tested on the data contained in the data-file by the trained model sub-component 418. The data file may contain a further sub-set of the data relating to Latin American males aged 26 to 35 with diabetes which was not submitted in the initial data submission step in step S500. The data file may be generated by numerical simulation and then fed into the system 100 for the testing purposes.

The trained model is tested on the data file in a step S512 by the trained model sub-component 418 to see if the trained model makes predictions which are consistent with the historical data. This can be implemented using standard techniques for validating the accuracy of a trained model which has been trained using a machine learning approach as is described above.

If the trained model sub-component 418 determines that the predictions made by the trained model are correct, then the trained model sub-component 418 accepts the trained model in a step S516.

The evaluation and refinement component 406 will now be described with reference to FIGS. 6 and 7.

The evaluation and refinement component 600 comprises a model evaluation and interpretation sub-component 602 and a rule and model determination sub-component 604. The relationship between the model evaluation and interpretation sub-component 602 and the model determination sub-component 604 and the remaining parts of the artificial intelligence module 122 is also illustrated schematically in FIG. 4a.

After the trained model sub-component 418 accepts the trained model in step S516, the trained model sub-component transmits the data relating to the accepted trained model to the rule and model determination sub-component 604 in a step S700.

The data will comprise the details of the trained model. Reverting back to our example of the multi-variate normal distribution wherein the heart rate, respiratory rate, body temperature and urine output are each normally distributed, the data will identify the trained model in this way with the respective parameters of the respective normal distribution also transmitted.

The details of the respective danger zones would also be transmitted to the rule and model determination sub-component 604. The rule and model determination sub-component 604 then stores the details in local storage 606 in a step S702.

The rule and model determination sub-component 604 then uses the details of the trained model and the danger zone to generate a new file which contains rules associated to the trained model in step S704. The rules are then stored in local storage 606 with the trained model.

That is to say, the rule and model determination sub-component 604 determines rules based on the trained model and the danger zones and they are associated with a model.

In the example of Latin American males aged 26 to 35 with diabetes the rule and model determination sub-component 604 generates a set of rules which are based on the multi-variate normal distribution described above. That is to say, the thresholds on the parameters which are determined to characterise danger zones for the patient are converted into rules which are associated with Latin American males aged 26 to 35 with diabetes.

If the process of steps S500 to S516 related to white Caucasian females aged 55 to 65 with low blood pressure then these rules would likely be different as the trained model output by steps S500 to S516 would be different.

That is to say, the application of machine learning principles to the generation of trained models enables different models to be generated for patients who fit into different demographics and, subsequently, different rules to be generated for patients who fit into different demographics.

The example above indicated that the urine output of a Latin American male aged 26 to 35 with diabetes would be normally distributed as part of a multivariate normal distribution with the heart rate, respiratory rate and body temperature also forming the other normally distributed random variables. The example above also indicated there could be a linear relationship between the occurrence of shock and low urine output.

This means that the rules for a Latin American male aged 26 to 35 with diabetes can be adjusted if it is found they are experiencing shock. The effect being that if the rule said that the danger zone on urine output for a Latin American male aged 26 to 35 with diabetes indicated that a patient was deteriorating if urine output fell below 25 ml per minute, say, then, if the patient was experiencing shock, the danger zone could be adjusted to an indication the patient was deteriorating if urine output fell below 20 ml per minute.

A multitude of other examples are possible. If the data input in step S500 related to patients who were suffering from sepsis then the rules would likely be different. The rules would likely indicate that careful monitoring of fluid output was required. By comparison to a Latin American male aged 26 to 35 with diabetes suffering from shock, the rule on urine output for a male individual aged 26 to 35 suffering from sepsis would likely be quite different as sepsis requires more stringent monitoring than diabetes.

Another example could be where the data relates to patients with heart failure which would lead to a different trained model.

Another example could be where the data relates to patients with chronic kidney disease and so produce less urine than a normal person, i.e. a person without chronic kidney disease. This could lead to the determination of a different trained model where the thresholds on urine output were adjusted appropriately to reflect the symptoms of chronic kidney disease.

That is to say, the trained models form a knowledge base for the system 100 and this knowledge base is built on when more training data is added to the system 100.

The rules determined in step S704 are then transmitted to the model evaluation and interpretation sub-component 602 in step S706.

The model evaluation and interpretation sub-component 602 is configured to test the rules generated by the rule and model determination sub-component 604 against the national and local guidelines in a step S708. The national and local guidelines are programmed in as a series of rules and are fed into the model evaluation and interpretation sub-component 602 from the clinical guidance sub-component 610.

Referring back to our previous example of Latin American males aged 26 to 35 with diabetes, if the national and local guidelines state that a specific urine output over a specific period of time is a sign of danger for a person within this demographic and the rules determined in step S704 do not indicate such a danger zone, then the rules are either refined to bring consistency to the rules (if the rules are providing outcomes that are sufficiently close to the national and local guidelines), i.e. the danger zones are refined or they are rejected and the model evaluation and interpretation sub-component 602 transmits a request back to the pre-processing and data transformation sub-component 602 for a revised trained model and rules. This re-initialises the steps S500 to S516 and the steps S700 to S708 to generate a revised training model and rules.

That is to say, the rules are expertly assessed by the model evaluation and interpretation sub-component and if the rules do not offer consistency with the national and local guidelines then they are rejected and the processes illustrated in FIGS. 5 and 7 are repeated until the rules do offer consistency with the national and local guidelines.

Step S708 may also involve human input wherein a health expert or an expert in a particular field of health assesses the rules and what they infer. The human can then indicate to the system that the rules are consistent with national and local guidelines or not.

The steps that may be taken by the model evaluation and interpretation sub-component 602 are illustrated in FIG. 6a.

The rules and the trained model can be fed into an input interface 800 which initialises a quality checking module 802 which is configured to enable the health practitioner to check the completeness, uniqueness, timeliness, validity, accuracy and consistency of the model and the rules.

If the model and the rules fail the quality checking tests initialised by quality checking module 802, then the model and rules are rejected and steps S500 to S512 and S700 to S708 are repeated until the model and rules do pass the quality checking tests.

The rules and the model are then fed into an evidence checking module 804 which is configured to enable the health practitioner to determine the conformity between the guidelines and the model and rules.

If the conformity between guidelines and the model and rules is indicated by evidence checking module 804 then the model and rules are accepted then it is determined that the rules offer consistency with the national and local guidelines then step S710 is initialised as detailed below.

If the conformity between the guidelines and the model cannot be determined then further investigation is required and the model and rules are rejected and steps S500 to S512 and S700 to S708 are repeated until the model and rules do conform to the national and local guidelines after further investigation.

The further investigation may involve obtaining more data.

If the test conducted in step S708 determines that the rules offer consistency with the national and local guidelines then the rules are added to the knowledge base for that demographic in a step S710. Referring back to our example of Latin American males aged 26 to 35 with diabetes the rules and the trained model become part of the knowledge base for that demographic.

The model evaluation and interpretation sub-component 602 may also be used to revise the knowledge base and to re-evaluate and re-interpret the information and rules that already form part of the knowledge base.

The national and local guidelines also form part of the knowledge base which is built by the system. These guidelines provide “simple” rules for common situations that occur in the medical treatment environment. The identification of more complex rules relating to specific demographic groups is only achieved through the application of the machine learning approach described above.

The training data which is supplied to the system 100 can be constantly updated based on updates to the data and updates to treatments and the knowledge base around those treatments.

This enables more complex rules to be programmed into the knowledge base based on research in the laboratory. For example, if it is found that, for example, the urine output of a patient experiencing shock can be improved by administering a new treatment then this may be fed into the data and the training data improved by the update.

The output from the machine learning approach may identify other rules.

For example, if the patient has sepsis then the rules will indicate more careful fluid monitoring is necessary than for somebody without sepsis.

In another example, if the patient has chronic kidney failure then the rules for urine output will be adjusted in that the expected range of urine output would be adjusted and, resultantly, the danger zones would also be adjusted. That said, if a more complex rule was established around patients with chronic kidney failure which emphasised danger zones on heart rate and respiratory rate then the rules may indicate the necessity for action anyway, i.e. even if the urine output is not in the danger zone.

An example of a simple rule is given in the table below:

TABLE 1
Example of a simple rule

| Urine Output/hour | Action | Monitoring |
|---|---|---|
| Greater than 30 ml | No action necessary | 1 hourly |
| Less than 30 ml | Alert practitioner | 30 minute intervals |
| Less than 25 ml | 250 bolus Hartmans | 30 minute monitoring |
| | solution | |

The simple rule described in Table 1 could be used in instances of sepsis where dropping urine output necessitates action. The actions to be taken have been indicated as appropriate by the application of machine learning principles to training data related to patients with sepsis.

However, if the patient was suffering with shock, the simple rule could look like this:

TABLE 2
Example of the adjustment of the rule in Table 1

| Urine Output/hour | Action | Monitoring |
|---|---|---|
| Greater than 25 ml | No action necessary | 1 hourly |
| Less than 25 ml | Alert practitioner | 30 minute intervals |
| Less than 20 ml | 250 bolus Hartmans | 30 minute monitoring |
| | solution | |

Sepsis and shock are ubiquitous concerns for the medical environment so such rules could form part of the knowledge base without the need for a priori application of the machine learning techniques described above.

We now describe with reference to FIG. 8 how the artificial intelligence module 122 generates an alert responsive to an input from the system 100 using the determined rules and trained model.

The flow of urine into the measuring container 106 from the patient causes the urine to fill the measuring container 106 where the urine is stored for measurement and analysis by the system 100. Deterioration in the patient's medical condition may cause the urine output to change, i.e. it can increase or decrease.

The system 100 comprises a user interface on the touch-screen display 140 which enables the medical practitioner to retrieve the patient's electronic health record which contains details of the patient's medical history and their demographic position. The electronic health record also indicates the treatments that the patient is undergoing. Information about the patient enables operatives of the system 100 to retrieve the correct trained models which ensures the knowledge base is used correctly or at least the best it can be.

The operative of the system 100 can assign a practitioner to the patient. The practitioner has a mobile device which can receive messages from the system 100. The practitioner may also designate other practitioners who can also receive messages in respect of the patient.

This information is transmitted to the artificial intelligence module 122 via the interface 404 in a step S900. The artificial intelligence module 122 retrieves the rules and the trained model for the patient, i.e. the rules and model for Latin American males aged 26 to 35 with diabetes are retrieved from storage and an alert generation sub-component is initialised with the rules and model for Latin American males aged 26 to 35 with diabetes. If any additional information relating to the patient is entered, i.e. that the patient is experiencing shock, then the rules and model for patients experiencing shock are also loaded into the alert generation sub-component.

In this instance, i.e. where the patient is a Latin American male aged 29 with diabetes and suffering from shock, the danger zone on the urine output would be adjusted accordingly as there is a link between urine output and shock. That is to say the danger zone on the urine output is refined as the knowledge base of the system 100 includes a rule about urine output and shock.

The artificial intelligence module 122 is also receiving input in the form of the patient's heart rate (beats per minute), blood pressure (millimetres of Mercury), body temperature (degrees Celsius), oxygen saturation of blood (percentage), respiratory rate (breaths per minute) and the time over which the patient has been connected to the system in seconds.

The processor 114 which determines the urine volume transmits a value indicative of the urine volume in the reservoir, the time interval over which the urine volume has been generated, the urine composition (in milliequivalents per litre (mEq/L) and the device orientation in degrees in a step S902. The transmittal of this information is repeated every second.

The system 100 may additionally include other sensors which determine urine composition. These sensors may indicate urine pH, the presence of nitrites, white blood cells, red blood cells, glucose, ketones and specific gravity.

The artificial intelligence module 122 receives the information in a step S904 and transmits the information to the knowledge base sub-component 800.

The knowledge base sub-component 800 compares the received value of the urine volume to the rules for the patient in a step S906. If the urine volume falls within the danger zone then a message is generated by the knowledge base sub-component 800 in a step S908.

In the example with a patient with sepsis and shock, the rule in Table 2 would be used by the knowledge base sub-component 800. That is to say, if the urine output of the patient over the course of an hour dropped below 25 ml then a message would be generated to alert staff that closer monitoring is required.

The message is transmitted to the communication module 126 in a step S910 with a request for an alert to be generated or for an instruction to be generated for a connected medical device such as a fluid bolus mechanism or a mechanical ventilator. The connected medical device may then alter their delivery to the patient responsive to the instruction.

Responsive to receiving the request for the alert to be generated in step S910, the communication platform matches the patient to the responsible practitioner and an alert indicating the urine volume and other medical information is transmitted to the responsible practitioner in a step S912. The practitioner can then take action.

The alert would contain the urine output of the patient over the course of the previous hour and a request for the practitioner to increase the monitoring intervals to every 30 minutes.

If the urine output of the patient dropped below 20 ml then a different alert would be transmitted to the responsible practitioner in step S912. In accordance with the rule in Table 2, the alert would request that a 250 ml bolus Hartmans solution be administered. Steps S900 to S912 are repeated every second as the values received in step S900 are received.

The practitioner would then attend to the patient and administer the 250 ml bolus Hartmans solution to the patient using the fluid bolus module 124—in accordance with the guidance provided by the rule in Table 2.

A more complex rule could be provided by the knowledge base for Latin American males aged between 26 and 35 with diabetes. Such a rule is described in the table below:

TABLE 3
Complex rule for Latin American males
aged between 26 and 35 with diabetes

| Urine Output (U), Heart | | |
|---|---|---|
| Rate (H), Respiratory | | |
| Rate (R) | Action | Monitoring |
| U > 30 ml AND H < 50 | No action | Hourly |
| bpm AND R > 100 Bpm | | |
| U < 30 ml AND H < 50 | Alert HCA | 30 minute intervals |
| U < 20 ml AND H < 50 | 200 ml bolus | 30 minute intervals |
| AND R < 100 | | |

This would mean that an alert is generated if the urine output dropped below 30 ml and the heart rate dropped below 50 bpm and an alert and a 200 ml bolus is recommended if the urine output dropped below 20 ml and the heart rate dropped below 50 bpm and the respiratory rate dropped below 100 Bpm.

Additionally, the complex rule could include the potential for the urine output to be normal and the input from other sources such as heart rate and respiratory rate to be indicative of a deterioration in the patient's condition.

Additionally, any of the rules in tables 1 to 3 could be adjusted if the patient was suffering from kidney failure as it is expected that urine output is lower.

After the alert has been generated, steps S900 to S912 are repeated.

The rules may be generated based on the knowledge base alone without input from the trained model. That is to say, the system 100 may have a stock of rules that indicate that a patient's condition is deteriorating without the requirement for further analysis of trained data. For instance, a change in blood pressure is typically an indicator of a change in health of a patient. A rule in the knowledge base may provide thresholds on the blood pressure which can be used to generate response actions the same as a particularly high or low urine output can.

Rules may be modified by any of the fields. One example may be where the fields relate to blood test results. That is to say, if the blood test results indicate an abnormal blood urea nitrogen level then this is an indicator of kidney function and this will likely influence what would be considered to be a normal level of urine output. If the system receives an input indicative of the patient having recent blood test results indicative of, say, abnormal blood urea nitrogen levels, then a rule from the knowledge base may impart a scaling on the rule generated using the trained model so that the danger zone is adjusted appropriately.

Rules may be modified by data related to imaging. Indeed, if the patient has recently had a CT scan with contrast, this can damage the kidneys which would change the normal levels or urine output. This may result in an alteration of the rule to request an increased level of monitoring if the urine drops below a specifically defined level.

We now describe, with reference to FIG. 9, the automated fluid bolus mechanism which is provided by the fluid bolus module 124.

The fluid bolus mechanism comprises a drip-stand 900 which can be moved along a supporting surface, i.e. a floor, on wheels (not shown). The drip-stand 900 is coupled to a container 902 which encloses a flexible bag 904 containing the fluid bolus to be delivered to the patient. In the example the fluid bolus is a 0.9% saline solution but it will be understood that any appropriate fluid could be contained in the bag 904. The container 902 has pressure sensitive points 906 along its sides to enable an operator to squeeze the bag 904 to provide a quick increase in the amount of fluid bolus being delivered to the patient. That is to say, a pressure cuff is provided to enable the bag to be squeezed to deliver a quick increase in the amount of fluid. The response action may indicate that a “quick” increase is what is needed.

The contents of the bag 904 leave the bag and enter syringe 908 which is configured to measure and convey a precise volume of fluid into the intravenous cannula 910.

In Table 1, Table 2 and Table 3, the rules described therein prescribe an action which is a delivery of an amount of fluid bolus. This is responsive to the urine output from the patient entering a danger zone which is determined by the trained model.

Where the urine output and the other measured parameters have entered the prescribed danger zone, an alert is generated in step S910. If the rule indicates that a delivery of an amount of fluid bolus is required, the communication module 126 may also transmit a request message to a communication interface 912 on the fluid bolus mechanism.

The communication interface 912 may be built into the container 902 where corresponding electronics 914 may be situated to receive the request message and convert the information in the request message into an action which correspondingly adjusts the amount of fluid bolus delivered to the patient through the cannula 910 by controlling the amount of fluid bolus which is conveyed through syringe 908.

The effect of this is that the fluid bolus is adjusted without the requirement for the practitioner to manually make the adjustments themselves.

The message to the fluid bolus mechanism may specify quantities, rates or specific fluid compositions to be delivered to the patient. The fluid bolus mechanism may comprise a plurality of containers 902 which each contain a different solution and each may be attached to a respective syringe 908 to control delivery of the respective solution. The message may specify different amounts of each solution at different rates and the automated fluid bolus mechanism ensures that the correct amount, as prescribed by the rule, is delivered.

The input to the fluid bolus mechanism is realised through the artificial intelligence module 122 and the machine learning principles applied to the training data.

The syringe 908 may be controlled by an electric motor which ensures that the syringe 908 only conveys the specified volume of fluid.

That is to say, the trained model generates a response from the system which causes something to happen, i.e. a response action is generated by comparing the values generated by the urine output of the patient with the rules that form part of the knowledge base.

The response action could be a message to a practitioner to increase or maintain monitoring or a message to a fluid bolus mechanism which causes the amount of fluid bolus delivered to the patient to be automatically adjusted.

The effect of this is that by applying the principles of machine learning and statistical inference to training data which is formed from clinical data and historical data for a specific demographic group, response actions can be “learned” from the data and used to inform practitioners about what is likely to be a successful course of action when a patient's condition deteriorates, i.e. the system is artificially intelligent and able to recommend actions based on what the analysis of the training data has determined.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. In the present specification, “comprises” means “includes or consists of” and “comprising” means “including or consisting of”. The singular reference of an element does not exclude the plural reference of such elements and vice-versa. The mere fact that certain measures are recited in mutually different dependent claims does not indicate that a combination of these measures cannot be used to advantage.

1. A healthcare monitoring system, the system arranged to receive at least one input parameter from at least one medical device, wherein the system is configured to infer a change in a patient's condition based on the at least one input parameter, wherein the system is further configured to infer the change in the patient's condition using a trained model.

2. The system of claim 1, wherein the system is configured to generate an alert based on the inference of a change in the patient's condition.

3. The system of claim 1, wherein the system is further arranged to receive a flow of biofluid from a biofluid source in or on the patient, the biofluid measurement system comprising:
a biofluid reservoir coupled to the biofluid source for receiving the flow of biofluid from the patient; and
at least one biofluid sensing arrangement configured to determine the volume of the biofluid in the biofluid reservoir to provide at least one output biofluid measurement value indicative of the volume of the biofluid in the biofluid reservoir;
wherein the system is configured to infer the change in the patient's condition based, at least in part, on the at least one output biofluid measurement value; and the system is further configured to generate an alert based on the at least one output biofluid measurement value.

4. The system of claim 3, wherein the biofluid sensing arrangement is further configured to determine values indicative of at least one of composition, temperature or other biometric of the biofluid in the biofluid reservoir.

5. The system of claim 2, wherein the system is further configured to communicate the alert to a device to enable a user to be notified of the inference of a change in the patient's condition.

6. (canceled)

7. (canceled)

8. (canceled)

9. The system of claim 3, wherein the biofluid sensing arrangement is configured to determine the volume of biofluid in the biofluid reservoir by weighing the biofluid reservoir.

10. (canceled)

11. (canceled)

12. (canceled)

13. (canceled)

14. (canceled)

15. (canceled)

16. (canceled)

17. (canceled)

18. (canceled)

19. (canceled)

20. The system of claim 1, wherein the system is configured to infer a change in the patient's condition using a knowledge base and rules determined using the knowledge base.

21. The system of claim 1, wherein the system is further configured to determine the trained model from the application of machine learning techniques to historical data, wherein the trained model is used to infer a change in the patient's condition.

22. The system of claim 1, wherein the system is further configured to determine the trained model from the application of statistical inference techniques to historical data, wherein the trained model is used to infer the change in the patient's condition.

23. (canceled)

24. The system of claim 18, wherein the fields pertain to a demographic group.

25. (canceled)

26. The system of claim 1, wherein the system is configured to determine at least one rule from the trained model.

27. The system of claim 26, wherein the rule defines a danger zone on at least one parameter, wherein the danger zone defines a threshold on the at least one parameter to indicate when a measurement for that parameter indicates deterioration in the patient.

28. (canceled)

29. The system of claim 22, wherein the system is configured to infer a change in the patient's condition responsive to a determination that the at least one parameter is indicating deterioration in the condition of the patient.

30. The system of claim 23, wherein, responsive to the system inferring a change in the patient's condition, the system is configured to initiate a response action designated by the rule.

31. The system of claim 27, wherein the response action is an instruction to a connected device.

32. The system of claim 30, wherein the response action is a message to a remote device to indicate the need for an increased frequency of monitoring of the patient.

33. (canceled)

34. The system of claim 30, wherein the response action is a request to a fluid bolus mechanism to increase or decrease the amount of fluid delivered to the patient.

35. (canceled)

36. The system of claim 34, wherein the fluid bolus mechanism, responsive to receiving the request, implements the request and delivers the requested amount or rate to the patient.

37. (canceled)

38. (canceled)

39. (canceled)

40. The system of claim 32, wherein the remote device is selected by the trained model based on the responsiveness of the operator designated to the remote device.

41. The system of claim 30, wherein the response action is a message to a remote device to indicate the need to decrease a currency frequency of monitoring of the patient.
